(19) United States
(12) Patent Application Publication
(10) Pub. No.: US 2008/0226575 A1
(43) Pub. Date: Sep. 18, 2008

(54) Long-Lasting Cosmetic Composition, Method For Using, and Process For Making

(75) Inventor: Fifi G. Hanna, Kearny, NJ (US)

Correspondence Address: AVON PRODUCTS, INC., AVON PLACE, SUFFERN, NY, 10901, US

(73) Assignee: Avon Products, Inc., Suffern, NY (US)

(21) Appl. No.: 12/066,299

(22) PCT Filed: Sep. 30, 2005
(86) PCT No.: PCT/US05/35042

§ 371 Date: Mar. 10, 2008

(51) Int. Cl. A61K 8/92; A61K 31/575; A61Q 1/10

(52) U.S. Cl. 424/70.7; 514/182; 424/70.1

(57) ABSTRACT

There is a cosmetic composition that has an anhydrous gel phase having a gellant and an organic solvent, an aqueous phase having a hydrophilic film former and water, and a wax phase having a wax and a hydrophilized lanolin derivative. The anhydrous gel phase, the aqueous phase, and the wax phase are substantially homogeneously admixed. The hydrophilized lanolin derivative is present in an amount sufficient to adsorb the aqueous phase. There is also a method of imparting curl, length, thickness, and/or color to eyelashes. There is further a process for making a cosmetic composition.

### BACKGROUND OF THE INVENTION

1. Field of the Invention

The present invention relates to a cosmetic composition that provides long lasting wear and excellent water resistance. More specifically, the present invention relates to a cosmetic composition, especially a mascara composition for eyelashes, that provides long-lasting wear, curl to the eyelashes, and exceptional water resistance. Most specifically, the present invention relates to compositions that provide a substantially waterproof film on the skin or lash.

2. Description of the Related Art

Mascara compositions are commonly employed by women to highlight and enhance the appearance of eyelashes. Such enhancements may include the imparting of color or tone, volume, i.e., thickness and length, and curl. Mascara compositions for the eyelashes have been formulated in both aqueous and anhydrous forms.

Aqueous compositions typically incorporate a hydrophilic film former to impart curl to eyelashes, to volumize and lengthen the eyelash, and to improve wearability. A disadvantage of aqueous compositions incorporating a hydrophilic film former is that they are not long-lasting and are removed easily with water. This is a serious disadvantage when the eyelash is exposed to the elements, e.g., rain and humidity, or when swimming, washing, or bathing.

Anhydrous compositions typically have waxes present that function to add volume to the eyelashes and render the compositions waterproof. A disadvantage of anhydrous compositions is they do not impart a high degree of curl to the eyelashes and are susceptible to smearing.

It would be desirable to have a mascara composition that provided the benefits and advantages of both aqueous and anhydrous formulations. It would further be desirable to have a mascara composition that long-lasting wear, curl, and water resistance to the eyelashes.

### SUMMARY OF THE INVENTION

It is an object of the present invention to have a cosmetic composition that provides long-lasting wear resistance and exceptional water resistance.

It is an object of the present invention to have a mascara composition that provides long-lasting wear resistance and exceptional water resistance.

It is also object of the present invention to have a mascara composition that provides to the eyelashes an exceptional degree of curl, a volumizing effect, and lengthening.

It is further an object of the present invention to have a mascara composition that provides a high degree of water resistance.

According to these and other objects of the invention, there is provided a cosmetic composition, especially a cosmetic composition that is suitable for use as a mascara composition. The composition has an anhydrous gel phase having a gellant and an organic solvent, an aqueous phase having a hydrophilic film former and water, and a wax phase having a wax and a hydrophilized lanolin derivative. The anhydrous gel phase, the aqueous phase, and the wax phase are substantially homogeneously admixed. The hydrophilized lanolin derivative is present in an amount sufficient to adsorb the aqueous phase.

Further according to these and other objects of the invention, there is a method of imparting curl and/or color to eyelashes. The method has the step of topically applying to the eyelashes a mascara composition having a) an anhydrous gel phase having a gellant, an organic solvent, and a colorant or pigment; b) an aqueous phase having a hydrophilic film former and water; and c) a wax phase having a wax and a hydrophilized lanolin derivative. The anhydrous gel phase, the aqueous phase, and the wax phase are substantially homogeneously admixed. The hydrophilized lanolin derivative is present in an amount sufficient to adsorb the aqueous phase.

Still further according to these and other objects of the invention, there is provided a process for making a mascara composition. The process has the steps of a) mixing an organic solvent with a gellant to prepare an anhydrous gel; b) admixing a wax and a hydrophilized lanolin derivative at an elevated temperature to prepare a molten wax phase; c) admixing the molten wax phase and the gel phase to prepare a gel/wax mixture; d) admixing a hydrophilic film former and water to prepare an aqueous phase; e) optionally admixing fibers with the aqueous phase; and f) admixing the aqueous phase and the gel/wax mixture at an elevated temperature to form the composition.

According to these and other objects of the invention, there is provided a mascara composition. The composition has an organic solvent, a gellant for the organic solvent, a hydrophilic film former, water, a wax, and a hydrophilized lanolin derivative, all of which are substantially homogeneously admixed. The hydrophilized lanolin derivative is present in an amount sufficient to substantially adsorb the water and the hydrophilic film former.

According to these and other objects of the invention, there is a cosmetic composition. The composition has an organic solvent, a gellant for the organic solvent, a hydrophilic film former, water, a wax, and a hydrophilized lanolin derivative, all of which are substantially homogeneously admixed. The hydrophilized lanolin derivative is present in an amount sufficient to substantially adsorb the water and the hydrophilic film former.

### DETAILED DESCRIPTION OF THE INVENTION

It was surprisingly found that there is a cosmetic composition providing long-lasting wear resistance and an exceptional degree of water resistance.

The cosmetic compositions of the present invention have in fact been found to be substantially unremovable with soap and water and usually require use of an organic solvent-based cosmetic remover to remove the product from the skin or lash. A suitable product to remove the formulations of the present invention from the skin or from a lash is Perfect Wear Make-up Remover sold by Avon Products, Inc. Inasmuch as the product composition of the present invention is water resistant as defined above, the consumer can apply facial moisturizers to the skin or lash over the composition without smudging, rub-off, or smearing. Additionally, inclement weather, including high humidity, does not appreciably affect the compositions that have been applied to skin or lash.

Applicant has further found that the compositions of the present invention are long-wearing, and are retained on the skin or lash for at least ten hours, preferably about twelve hours or more, and most preferably about 16 hours or more. Accordingly, the compositions of the present invention meet the industry requirements for “all day wear”. Such compositions are especially preferred when used as a mascara composition.

Unless otherwise indicated all percentages set forth herein are percent by weight of the total composition based on the active amount of the constituent.

The cosmetic composition is substantially a homogeneous mixture of three premixes referred to hereinafter as an anhydrous gel phase, a wax phase, and an aqueous phase. The use of the term “phase” in describing each of the three premixes comprising the cosmetic composition is not intended to indicate that the cosmetic composition is a multiphase composition, but rather to indicate that each of the premixes has its own distinct compositional nature.

The anhydrous gel phase premix has an organic solvent and a gellant. The wax phase premix has a wax and a hydrophilized lanolin derivative. The aqueous phase premix has a hydrophilic film former and water. Each phase is separately prepared, the gel phase and the wax phase admixed, and the aqueous phase subsequently admixed with the foregoing admixture to form the substantially homogenous composition. By “substantially homogeneous” is meant that the composition of the present invention behaves as a single phase liquid, and does not appreciably clump, coagulate, or exude ingredients(s) used in its preparation during normal use conditions.

The composition enables a hydrophilic film former (oil resistant) and a wax to be used in combination with each other. The composition affords the advantages of both aqueous and anhydrous (wax-based) formulations, i.e., high degree of curl to eyelashes, added volume, and an exceptional degree of water resistance. The hydrophilic film former forms a film on the surface of eyelashes and imparts a desirable degree of curl to eyelashes. The hydrophilic film former is soluble or dispersible in water.

The hydrophilic film former is preferably selected from the group consisting of water-soluble cationic polymers, anionic polymers, and nonionic polymers. Useful hydrophilic film formers include, but are not limited to, acrylics (acrylates), polyacrylates, acrylamide polymers and copolymers and quaternary salts thereof, urethanes, polyurethanes, polyesters, polysaccharides, polyamides, polyimides, polyols, polyethers, cellulosics, proteins, polyamino acids, esters derived from rosin, latexes, and any combinations thereof.

Particularly preferred hydrophilic film formers are cationic polymers. Useful cationic polymers include, but are not limited to, hydroxymethyl chitosan, hydroxyethyl cellulose, Polyquaternium-4, Polyquaternium-5, Polyquaternium-6, Polyquaternium-7, Polyquaternium-10, Polyquaternium-22, and any combination thereof. Polyquaternium-7 is most preferred. Polyquaternium-7 is an ammonium salt of an acrylamide/dimethyl diallyl ammonium chloride copolymer and is sold by Ciba Specialty Chemicals under the tradename Salcare Super 7.

Suitable water-soluble film forming anionic polymers are acrylamides copolymer sold as Sepigel 501 by SEPPIC, acrylates/ethylhexyl acrylate copolymer, acrylates copolymer (for example, Aculyn 33, sold by Rohm & Haas), acrylates/vinylpyrrolidone copolymer, and sodium acrylates copolymer.

Suitable nonionic water soluble film forming polymers are Polyurethane-1, Polyimide-1, and Adipic Acid/PPG-10 copolymer. A preferred Polyimide-1 is Aquaflex by ISP.

The hydrophilic film former is present in an amount sufficient to provide for film formation on the surface of the skin or eyelashes and to impart a desirable degree of curl to eyelashes. Preferably, the hydrophilic film former is present from about 0.1 wt % to about 5 wt %. More preferably, the hydrophilic film former is present from about 0.1 wt % to about 2 wt %. Most preferably, the hydrophilic film former is present from about 0.1 wt % to about 1 wt % based upon the total weight of the composition.

The cosmetic composition has one or more waxes therein. Suitable waxes include any known in the cosmetic art, such as those of animal origin, plant origin, mineral origin, and synthetic origin. Suitable waxes include, but are not limited to, rice bran wax, carnauba wax, ouricurry wax, candelilla wax, montan wax, sugar cane wax, ozokerite wax, polyethylene wax, beeswax, microcrystalline wax, or any combination thereof.

Suitable waxes have a melting point in the range from about 60° C. to about 110° C. and preferably from about 700 to about 90° C. and a needle penetration in the range from about 1 to about 40, preferably from about 3 to about 25, and most preferably from about 5 to about 20. The needle penetration of waxes is determined according to ASTM D 1321. Mixtures of hard and soft waxes are preferred to provide a composite melting point and needle penetration within the preferred ranges. A mixture of carnauba wax, beeswax, ozokerite, and microcrystalline wax is most preferred.

The wax is present in the composition in an amount effective to enhance the volume of eyelashes and to impart water resistance to the composition. Preferably, the wax is present at about 2 wt % to about 35 wt % based on the total weight of the composition. More preferably, the wax is present at about 5 wt % to about 30 wt %. Most preferably, the wax is present at about 10 wt % to about 20 wt % based on the total weight of the composition.

The composition has a hydrophilized lanolin derivative. The hydrophilized lanolin derivative is sufficiently hydrophobic such that it is miscible with the (molten) wax when the two are admixed. On the other hand, the lanolin derivative is sufficiently hydrophilic such that it acts as adsorbing agent to sequester and retain the aqueous phase, including the hydrophilic film former, in the wax such that the aqueous phase is substantially dispersed as a discontinuous phase within the wax without phase separation. The hydrophilized lanolin derivative is lanolin that has been functionalized with a substituent(s) to increase its degree of hydrophilicity (relative to non-functionalized lanolin). The hydrophilized lanolin derivative is functionalized to a sufficient degree such that it can act as an effective adsorbing agent.

Useful hydrophilized lanolin derivatives include, but are not limited to, hydroxylated lanolin, hydrogenated lanolin, PEG-10 hydrogenated lanolin, acetylated lanolin, lanolin alcohol, lanolin fatty acid, PPG-2 lanolin ether, and any combination thereof. A preferred hydroxylated lanolin is OHlan sold by Amerchol Corporation.

The hydrophilized lanolin derivative is present in an amount sufficient to adsorb the aqueous phase. The amount of hydrophilized lanolin derivative needed will depend on such factors as the hydrophilicity of the lanolin derivative and the amount of aqueous phase to be adsorbed. Typically, the hydrophilized lanolin derivative is present from about 0.1 wt % to about 10 wt %. More typically, the hydrophilized lanolin derivative is present from about 0.5 wt % to about 5 wt %. Most typically, the hydrophilized lanolin derivative is present from about 1 wt % to about 4 wt % based upon the total weight of the composition.

The composition has a gellant. The gellant functions to form an anhydrous gel phase with an organic solvent in the composition. A preferred gellant is a clay. Useful clays include, but are not limited to, bentonite, hectorite, kaolin, and montmorillonite, all of which are widely commercially available. A particularly preferred gellant is disteardimonium hectorite, sold as Bentone 38V by Rheox. Suitable nonclay gellants include olefin/styrene copolymers, such as Versagel M and Versagel MC (available from Penreco) and Gel Base (available from Brooks Industries), and propylene carbonate.

The gellant is present in the composition in an amount sufficient to form an anhydrous gel phase with an organic solvent. The gellant is preferably present from about 0.5 wt % to about 20 wt % and most preferably present from about 2 wt % to about 5 wt %.

The organic solvent functions to form an anhydrous gel phase with the gellant. The organic solvent is also available in the finished composition to dissolve the wax component. Useful organic solvents include, but are not limited to, isododecane (for example, Permethyl 99A by Presperse, Inc.), isoparaffins (for example, the solvents sold under the tradename Isopar, hexadodecane, cyclomethicone, and combinations thereof.

The anhydrous gel phase present in the composition provides a waterproof coating on the eyelash or the skin that exhibits exceptional water resistance or waterproofness. When applied, the composition is in the form of a gel. But as solvent evaporates, including both water and the organic solvent, a waxy film is left on the eyelash or skin. The waxy film, together with the water soluble film forming hydrophilic polymer, provide a film that is highly resistant to removal by water and very wear resistant. When used as a mascara, the composition provides a substantial volumizing and curling benefit due to the retraction of the film former upon evaporation of the solvent.

The organic solvent has a flash point of at least 30° C., preferably from about 35° C. to about 90° C., and most preferably from about 40° C. to about 60° C. (as measured by ASTM D6450 or ASTM D93).

The organic solvent is present from about 5 wt % to about 80 wt % and more preferably from about 20 wt % to about 50 wt % based on the total weight of the composition.

The composition preferably has a water content in the composition of from about 0.5 to about 25 wt %. More preferably, the composition has a water content of about 15 wt % or less based on the total weight of the composition. The water is used as a solvent or diluent for the hydrophilic film former. The hydrophilic film former typically provides soft flexible films that exhibit good retraction, and hence are able to curl the lash upon evaporation of the water (and other volatiles).

The cosmetic composition has one or more pigments, pearlescents, and/or colorants therein (terms used interchangeably). Useful pigments and/or colorants include any known in the cosmetic art. Coatings and surface treatments may also enhance the shine or gloss exhibited by mascara compositions. Examples of useful pigments include titanium dioxide, zinc oxide, iron oxide, chromium oxide, ferric blue, and mica; organic pigments include barium, strontium, calcium or aluminum lakes, ultramarines, and carbon black; colorants include D&C Green #3, D&C Yellow #5, and D&C Blue #1. Pigments and/or colorants may be coated or surface treated with one or more compatibilizers to aid in dispersion in either or both of the aqueous or wax phases. Preferred pigments and/or colorants are those surface treated with dimethicone copolyol.

Pigments/pearlescents/colorants can be present in the composition in an amount of 1 to about 25 wt. % of the total weight of the composition, and preferably in an amount of about 1 to about 15 wt. % based on the total weight of the composition.

The composition optionally has one or more hydrophobic (water-resistant) film formers therein to aid in film formation. Hydrophobic film formers include, but are not limited to, polyolefins, polyvinylpyrrolidone polymers and copolymers, polyethylenes, polyalkyls, polystyrenes, triglycerides, epoxy resins, shellacs, or any combinations thereof. Preferred hydrophobic film formers are pentaerythrytol rosinate and polyvinyl pyrrolidone/eicosene copolymers. Some of the aforementioned waxes, such as beeswax, carnauba wax, ozokerite wax, and microcrystalline wax, also function as hydrophobic film formers. Hydrophobic film formers can be incorporated into the anhydrous gel phase and/or the wax phase.

The composition of the present invention optionally has a multiplicity of fibers therein. When the mascara composition is applied, the fibers are laid on the surfaces of the eyelashes. The fibers enhance the volume of the eyelashes, i.e., increase thickness and length thereof. The fibers may be of any type known in the cosmetic art and may be natural or synthetic. Thermoplastic fibers, such as those of polyethylene, polypropylene, or nylon, are preferred. The fibers can be colored or surface treated for visual effect. The fibers can be added into any of the anhydrous gel, wax, or aqueous phases but are preferably added in the aqueous phase.

The composition may have one or more optional ingredients. Useful optional ingredients include, but are not limited to, botanical extracts, chelating agents, depigmenting agents, emollients, firming agents, fragrances, humectants, gloss agents such as triisostearoyl polyglyceyl-3 dimer dilinoleate, moisturizers, preservatives, stabilizers, surfactants, and vitamins.

The cosmetic composition can take the form of a number of different products. Suitable products include, but are not limited to, mascara, eye liner, blushes, and lip colorants/glosses.

In the compositions of the present invention, the proportions of the anhydrous gel phase, the wax phase, and the anhydrous phase are maintained such that the final mascara composition is substantially a homogenous mixture of the three. In this regard, there is provided an aqueous phase with sufficient water to solublize the hydrophilic film former that provides the desired lash curling benefit of the mascara composition. The wax phase contains the hydrophilic lanolin, which has sufficient hydrophilicity to retain the aqueous phase homogeneously in the composition. The anhydrous gel phase provides the composition with its gel physical form. The wax phase together with the adsorbed aqueous phase provide a highly wear resistant and highly water resistant coating notwithstanding the presence of the hydrophilic film former.

The anhydrous phase comprises from 70 wt % to 95 wt % and preferably from about 78 wt % to 90 wt % of the composition based on the total weight of the composition. The wax phase comprises from about 4 wt % to about 25 wt % and preferably from about 10 wt % to about 20 wt %. The aqueous phase comprises from about 1 wt % to about 25 wt % and preferably from about 5 wt % to about 15 wt %.

Another feature of the present invention is the process for making the mascara composition. The process generally has the steps of forming an anhydrous gel, forming a wax phase, adding the anhydrous gel phase to the wax phase to form a gel/wax mixture, and adding an aqueous phase having the hydrophilic film former to the gel/wax mixture. The anhydrous gel is prepared by mixing one or more organic solvents with one or more gellants. Optionally, hydrophobic (water-resistant) film formers, fillers and volumizers, such as polymethylmethcrylate (PMMA), silica, and aluminum distearate, and colorants may be added to the anhydrous gel as well. The wax phase is prepared by mixing one or more waxes and the hydrophilized lanolin derivative at an elevated temperature, preferably about 170-175° F. Optionally, preservatives and gloss agents may also be added to the wax phase. The wax phase is added and mixed in molten form in the gel phase. The aqueous phase is prepared by mixing the hydrophilic film former with water. Optionally, fibers and fillers may be added to the aqueous phase. The aqueous phase is heated to an elevated temperature and added to the gel/wax mixture at an elevated temperature, preferably about 120° F. It is understood that the foregoing disclosed temperatures will vary depending on the physical properties of the wax(es).

The following are examples of the present invention.

### EXAMPLES

| | Composition | | |
|---|---|---|---|
| | 1 | Composition 2 | Composition 3 |
| Components | (wt %)^{a,b} | (wt %)^{a,b} | (wt %)^{a,b} |
| ANHYDROUS PHASE | | | |
| Isododecane | 60 | 50 | 35 |
| Pentaerithrytol | 5 | 6 | 8 |
| rosinate | | | |
| Bentone 38V¹ | 1 | 2 | 5 |
| Propylene carbonate | 1 | 1.2 | 2 |
| Silica | 5 | | 2 |
| PMMA | | 10 | |
| WAX PHASE | | | |
| Carnauba wax | 5 | 4 | 5 |
| Beeswax | 5 | 4 | 4 |
| Ozokerite wax | 2 | 1.5 | 4 |
| Microcrystalline wax | 1 | 1.5 | 3 |
| Preservative | 0.9 | 0.9 | 0.9 |
| Hydroxylated lanolin² | 4 | 3.6 | 5 |
| Pigments | 4 | 6 | 6 |
| Fiber (Nylon) | 0.5 | 0.9 | 0.8 |
| TPDD⁶ | 1.5 | 1.5 | 1.5 |
| Hydrogenated | 1.2 | 1.2 | 1.2 |
| polydecene⁷ | | | |
| AQUEOUS PHASE | | | |
| Hydrophilic polymer | 1.2³ | 0.5⁴ | 0.8⁵ |
| Distilled water | Q.S. | Q.S. | Q.S. |
| | 100% | 100% | 100% |

^{a}Percentages are recited on an “as received” basis, with activity of the as received material identified in the footnotes. If not indicated activity is 100%.
^{b}Percentages by weight of the total composition.
¹Disteardimonium hectorite from Elementis Specialties, Inc.
²OHlan from Amerchol Corp.
³Polyimide-1 available as Aquaflex XL-30 from ISP (30% active aqueous dispersion).
⁴Polyquaternium-7 available as Salcare Super 7 from Ciba Specialty Chemicals (30% active).
⁵Acrylates Copolymer available as Daitosol 5000 AD from Daito Kasei (30% active aqueous dispersion).
⁶Triisostearoyl polyglyceryl-3 dimer dilinoleate sold by Scher Chemicals as Schercemol PTID.
⁷Ceraflow E by Shamrock Technologies, Inc.

The propylene carbonate solvent is heated to about 170-175° F. and the Bentone 38V clay and pentaerythrytol rosinate mixed in. Pigments, fillers (silica and PMMA) are then incorporated to obtain the anhydrous gel phase premix. The several waxes are heated to above their melting points to about 75-80° C., and preservative added, along with hydroxylated lanolin, TPDD hydrogenated polydecene, with mixing. The wax phase premix is then added at this temperature to the anhydrous gel phase premix. The mixture is cooled to 65-70° C. The aqueous phase is prepared by heating water and the hydrophilic film forming polymer to about 65-70° C., and is added to the mixture, with mixing for 10 minutes. The mixture is then cooled to room temperature.

The above compositions can be applied to the lashes to impart curl, length, thickness, and color.

It should be understood that the foregoing description is only illustrative of the present invention. Various alternatives and modifications can be devised by those skilled in the art without departing from the invention. Accordingly, the present invention is intended to embrace all such alternatives, modifications and variances that fall within the scope of the appended claims.

What is claimed is:

1. A cosmetic composition, comprising:
an anhydrous gel phase having a gellant and an organic solvent;
an aqueous phase having a hydrophilic film former and water;
a wax phase having a wax and a hydrophilized lanolin derivative,
wherein the anhydrous gel phase, the aqueous phase, and the wax phase are substantially homogeneously admixed, and wherein the hydrophilized lanolin derivative is present in an amount sufficient to adsorb the aqueous phase.

2. The composition of claim 1, wherein the hydrophilic film former is selected from the group consisting of cationic polymers, anionic polymers, and nonionic polymers.

3. The composition of claim 2, wherein the hydrophilic film former is a cationic polymer.

4. The composition of claim 3, wherein the cationic polymer is selected from the group consisting of Polyquaternium-4, Polyquaternium-5, Polyquaternium-6, Polyquaternium-7, Polyquaternium-10, Polyquaternium-22, and any combination thereof.

5. The composition of claim 2, wherein the hydrophilic film former is an anionic polymer selected from the group consisting of acrylamides copolymer, acrylates/ethylhexy acrylate copolymer, acrylates copolymer, acrylates/vinylpyrrolidone copolymer, sodium acrylates copolymer, and any combination thereof.

6. The composition of claim 2, wherein the hydrophilic film former is a nonionic polymer selected from the group consisting of Polyurethane-1, Polyimide-1, Adipic Acid/PPG-10 copolymer, and any combination thereof.

7. The composition of claim 1, wherein the hydrophilic film former is present from about 0.1 wt % to about 2 wt % based upon the total weight of the composition.

8. The composition of claim 1, wherein the hydrophilic film former is present from about 0.1 wt % to about 1.5 wt % based upon the total weight of the composition.

9. The composition of claim 1, wherein the hydrophilic film former is present from about 0.1 wt % to about 1 wt % based upon the total weight of the composition.

10. The composition of claim 1, wherein the hydrophilized lanolin derivative is selected from the group consisting of hydroxylated lanolin, hydrogenated lanolin, acetylated lanolin, PEG-10 hydrogenated lanolin, acetylated lanolin, lanolin alcohol, lanolin fatty acid, PPG-2 lanolin ether, and any combination thereof.

11. The composition of claim 10, wherein the hydrophilized lanolin derivative is hydroxylated lanolin.

12. The composition of claim 1, wherein the hydrophilized lanolin derivative is present from about 0.1 wt % to about 10 wt % based upon the total weight of the composition.

13. The composition of claim 1, wherein the hydrophilized lanolin derivative is present from about 0.5 wt % to about 5 wt % based upon the total weight of the composition.

14. The composition of claim 1, wherein the hydrophilized lanolin derivative is present from about 1 wt % to about 4 wt % based upon the total weight of the composition.

15. The composition of claim 1, wherein the wax is selected from the group consisting of beeswax, carnauba, microcrystalline, ozokerite, and combinations thereof.

16. The composition of claim 1, wherein the wax is present from about 8 wt % to about 35 wt % based upon the total weight of the composition.

17. The composition of claim 1, further comprising a colorant or pigment.

18. The composition of claim 1, further comprising fibers.

19. The composition of claim 1, wherein the gellant is a clay.

20. The composition of claim 19, wherein the clay is selected from the group consisting of bentonite, hectorite, kaolin, and montmorillonite.

21. The composition of claim 1, wherein the gellant is present from about 0.5 wt % to about 20 wt % based on the total weight of the composition.

22. The composition of claim 1, wherein the organic solvent is selected from the group consisting of isododecane, propylene carbonate, isoparaffin and combinations thereof.

23. The composition of claim 1, wherein the organic solvent is present from about 5 wt % to about 80 wt % based on the total weight of the composition.

24. A method of imparting curl and/or color to eyelashes, comprising topically applying to the eyelashes a mascara composition, having
a colorant or pigment;
an anhydrous gel phase having a gellant and an organic solvent;
an aqueous phase having a hydrophilic film former and water; and
a wax phase having a wax and a hydrophilized lanolin derivative,
wherein the anhydrous gel phase, the aqueous phase, and the wax phase are substantially homogeneously admixed, and wherein the hydrophilized lanolin derivative is present in an amount sufficient to adsorb the aqueous phase.

25. A process for making a mascara composition, comprising:
a) mixing an organic solvent with a gellant to prepare an anhydrous gel;
b) admixing a wax and a hydrophilized lanolin derivative at an elevated temperature to prepare a molten wax phase;
c) admixing the molten wax phase and the gel phase to prepare a gel/wax mixture;
d) incorporating a pigment or colorant in any of steps a, b or c.
e) admixing a hydrophilic film former and water to prepare an aqueous phase;
f) optionally admixing fibers with the aqueous phase;
g) admixing the aqueous phase and the gel/wax mixture at an elevated temperature;
h) cooling the admixture obtained in step g.

26. A cosmetic composition, comprising:
an organic solvent;
a gellant for the organic solvent;
a hydrophilic film former;
water;
a wax; and
a hydrophilized lanolin derivative,
wherein the organic solvent, the gellant, the hydrophilic film former, the water, the wax, and the hydrophilized lanolin derivative are substantially homogeneously admixed, and wherein the hydrophilized lanolin derivative is present in an amount sufficient to substantially adsorb the water and the hydrophilic film former.
